(19) United States
(12) Patent Application Publication
(10) Pub. No.: US 2003/0199488 A1
(43) Pub. Date: Oct. 23, 2003

(54) Treatment of hyperproliferative disorders/inflammatory dermatoses

(76) Inventor: Gina M. Trotta, Wilmington, NC (US)

Correspondence Address: WILLIAM J. MASON, MACCORD MASON PLLC, POST OFFICE BOX 1489, WRIGHTSVILLE BEACH, NC, 28480, US

(21) Appl. No.: 10/420,255

(22) Filed: Apr. 22, 2003

Related U.S. Application Data
(60) Provisional application No. 60/375,173, filed on Apr. 23, 2002.

(51) Int. Cl. A61K 31/573; A61K 33/32; A61K 33/26; A61K 33/24; A61K 35/78

(52) U.S. Cl. 514/179; 424/647; 424/655; 424/642; 424/769; 424/776

(57) ABSTRACT

Hyperproliferative disorders and inflammatory dermatoses, in particular psoriasis, are treated with a composition comprised of an admixture of a corticosteroid in a pharmaceutically accepted carrier, in combination with a moist, non-viscous, semi-solid gelatinous paste. The treated area is then covered with a non-permeable occlusive.

This application claims the benefit of the filing date of Provisional Application Serial No. 60/375,173, filed Apr. 23, 2002.

### BACKGROUND OF THE INVENTION

(1) Field of the Invention

The present invention relates to compositions useful in the treatment of hyperproliferative disorders/inflammatory dermatoses, in particular psoriasis, and to the method of using such compositions.

(2) Description of the Prior Art

Psoriasis is a chronic persistent skin disorder consisting of hyperproliferating lesions wherein skin cells multiply up to ten times faster than normal. Psoriasis affects 2.6% of the population or more than 7 million people with approximately 150,000 to 260,000 new cases being diagnosed every year. Four hundred people die annually due to complications as a result of psoriasis.

There are a number of different treatments available to include topical and systemic applications with some being successful for temporary periods of time. The present treatment modalities currently available for the treatment of psoriasis have side effects that are severe and long term. Due to the fact that psoriasis is a chronic disease requiring ongoing treatment, a favorable treatment regime is one that is capable of alleviating active lesions for extended periods of time utilizing a safe and convenient methodology.

Disclosures in the prior art deal with unique combinations of various compounds effective in the treatment of hyperproliferative disorders. Many of these compositions are based on the use of corticosteroids. A corticosteroid can be any of a number hormonal steroid substances obtained from the cortex of the adrenal gland. They are classified according to their biological activity as glucorticoids, mineralcorticoids, and androgen. Andrenal corticosteroids do not initiate enzymatic and cellular activity, however they do permit many biochemical reactions to occur at optimal rates. Corticosteroids are pharmacologically unique in their composition and mechanism of action from retinoids which can have serious side effects, and beta-adrenergic receptor stimulating compounds which act on beta-receptors allowing sympathetic nervous transmissions to be stimulated.

It is known that the extent of percutaneous absorption of topical corticosteroids is determined by many factors including the vehicle, the integrity of the epidermal barrier, and the use of occlusive dressings. Occlusive dressings substantially increase the percutaneous absorption of topical corticosteroids. Thus, occlusive dressings may be a valuable therapeutic adjunct for treatment of resistant dermatoses. The following patents illustrate prior art compositions and methods proposed to treat hyperproliferative disorders:

U.S. Pat. No. 4,038,417 to Nelson describes a method for treating psoriasis using a beta-adrenergic receptor stimulating compound together with a topical pharmaceutical carrier. The solution or ointment is generally applied for five days, preferably using a continuous occlusive dressing.

U.S. Pat. No. 4,190,669 to Vorhees et al. describes a method for treating psoriasis in which a composition of eicosa-5,8,11,14-tetraynoic acid, its congeners, and the lower alkyl esters thereof and a pharmaceutical carrier. The composition may be applied topically or by injection or orally. It was found that topical application with an occlusion produced improved results.

U.S. Pat. No. 5,998,395 to Klingman describes a method for treating psoriasis using a composition containing both a corticosteroid and a retinoid. Applications are administered once or twice daily. In general it is known that individual topical corticosteroids vary in potency and efficacy.

U.S. Pat. No. 5,658,559 to Smith describes a method for treating pathologies of the skin utilizing an occlusive or semi-occlusive barrier moisturizing lotion. When the lotion is applied to the skin and dries, a polymeric film forms.

Despite numerous prior art efforts to address this problem, there is still a need for a safe and easy treatment regime that is successful in alleviating active lesions for extended periods of time and/or remission of such lesions.

### SUMMARY OF THE INVENTION

It is known to treat psoriasis with a corticosteroid, normally dispersed in a pharmaceutically accepted vehicle/carrier that usually consists of an oil, lotion or cream and may contain a skin penetrating enhancer, and to cover the treatment area with an occlusive dressing. In accordance with the present invention, it has been found that inclusion of an additional ingredient, namely a moist, non-viscous, semi-solid gelatinous paste, in the treatment composition, substantially improves the results achieved.

The paste ingredient may contain specific active ingredients or simple materials such as oils, waxes and starch and also removes metabolic wastes and impurities. As an essential requirement, the paste must not dissipate or completely absorb into the skin for a minimum of 48 hours with the use of an occlusive.

Thus, the present formulation is comprised of a corticosteroid, normally dispersed in a pharmaceutically accepted vehicle/carrier that usually consists of an oil, lotion or cream and may contain a skin penetrating enhancer, in combination with a paste having the above characteristics. These ingredients may be combined prior to application, or applied separately.

This composition is applied to the affected area, and covered with a non-permeable occlusive placed over the active lesion and remains for 72 to 96 hours and to be removed daily for dressing changes. The combination of the aforementioned components and their method of application are critical to the results.

Accordingly, it is an object of the present invention to provide a unique composition wherein one of the four critical elements is a compound that is a non-viscous, semi-solid gelatinous paste that may contain specific active ingredients or simple materials such as oils, waxes and starch and also removes metabolic wastes and impurities and does not dissipate or completely absorb into the skin with the use of an occlusive for a minimum of 48 hours. The compound referred to is unique and is not equivalent to a lotion composition which is liquid in form and is comprised primarily of water and emolients.

These and other aspects of the present invention will become apparent to those skilled in the art.

### DETAILED DESCRIPTION OF THE INVENTION

Efficacy of this invention is dependent on multiple factors whereas the four components described along with the method of application resulted in unexpected results i.e.; remission of the majority of lesions with reoccurrence of a small percentage. The invention relates to a pharmaceutical composition, or system, for topical administration comprising of:

A) A corticosteroid;

B) A pharmaceutically accepted vehicle containing an oil, cream, lotion, jelly etc. and may contain a penetrating enhancer;

C) A composition that is comprised of a moist, non-viscous, semi-solid gelatinous paste that may contain specific active ingredients or simple materials such as oils, waxes, and starch and also removes metabolic wastes and impurities and must not dissipate or completely absorb into the skin for a minimum of 48 hours with the use of an occlusive. Description of the preferred embodiments include: Mineral oil, Lanolin, Beeswax, Cetyl Esters, Zinc Oxide, Witch Hazel, Propylene Glycol, Cocoa Butter, Glycerin, SD Alcohol 40, Boric Acid, Petrolatum, Methylparaben, Propylparaben, Camphor, Benzoin, Chromium Hydroxide Green, Menthol, Ceresin and Iron Oxides; and

D) A non-permeable barrier/occlusive appropriately shaped and sized to cover the application of the admixed products applied to the lesions for 72 to 96 hour periods only to be removed daily for dressing changes and reapplication of the aforementioned components. Whereas the combined therapy is more efficacious than either ingredient or treatment modality alone.

It was found that removal of component C rendered only partially favorable results at best and by addition of component C rendered unexpected results i.e.: remission of the majority of lesions. The present invention provides substantial cost savings due to the reduction in the required amount of corticosteroid utilized in conventional therapies and is safer than some current conventional therapies available today. Furthermore, the invention provides remarkable therapeutic benefits that are expeditious and thereby reduce the risks associated with prolonged therapies involving conventional corticosteroid treatment regimes.

Certain modifications and improvements will occur to those skilled in the art upon a reading of the foregoing description. It should be understood that all such modifications and improvements have been deleted herein for the sake of conciseness and readability but are properly within the scope of the invention.

What is claimed is:

1. A composition useful in the treatment of hyperproliferative disorders and inflammatory dermatoses comprised of:
a) a corticosteroid; and
b) a moist, non-viscous, semi-solid gelatinous paste.

2. The composition of claim 1, wherein said corticosteroid is dispersed in a pharmaceutically accepted carrier.

3. The composition of claim 1, further including a skin penetrating enhancer.

4. The composition of claim 1, wherein said paste will not dissipate or be completely absorbed into the skin for a minimum of 48 hours.

5. The composition of claim 1, wherein said paste includes at least one ingredient selected from the group consisting of mineral oil, lanolin, beeswax, cetyl esters, zinc oxide, witch hazel, propylene glycol, cocoa butter, glycerin, sd alcohol 40, boric acid, petrolatum, methylparaben, propylparaben, camphor, benzoin, chromium hydroxide green, menthol, ceresin and iron oxides.

6. A system useful in the treatment of hyperproliferative disorders and inflammatory dermatoses over an affected area comprised of:
a) a composition including corticosteroid and a moist, non-viscous, semi-solid gelatinous paste; and
b) a non-permeable occlusive to cover said composition and said affected area.

7. The composition of claim 6, wherein said corticosteroid is dispersed in a pharmaceutically accepted carrier.

8. The composition of claim 6, further including a skin penetrating enhancer.

9. The composition of claim 6, wherein said paste will not dissipate or be completely absorbed into the skin for a minimum of 48 hours.

10. The composition of claim 6, wherein said paste includes at least one ingredient selected from the group consisting of mineral oil, lanolin, beeswax, cetyl esters, zinc oxide, witch hazel, propylene glycol, cocoa butter, glycerin, sd alcohol 40, boric acid, petrolatum, methylparaben, propylparaben, camphor, benzoin, chromium hydroxide green, menthol, ceresin and iron oxides.

11. A method for treating psoriasis over an affected area comprised of:
a) applying to the affected area composition including a corticosteroid and a moist, non-viscous, semi-solid gelatinous paste; and
b) covering the affected area and said composition with a non-permeable occlusive.

12. The method of claim 11, wherein said corticosteroid is dispersed in a pharmaceutically accepted carrier prior to application to the affected area.

13. The method of claim 11, further including applying a skin penetrating enhancer to the affected area.

14. The method of claim 11, wherein said paste will not dissipate or be completely absorbed into the skin for a minimum of 48 hours.

15. The method of claim 11, wherein said paste includes at least one ingredient selected from the group consisting of mineral oil, lanolin, beeswax, cetyl esters, zinc oxide, witch hazel, propylene glycol, cocoa butter, glycerin, sd alcohol 40, boric acid, petrolatum, methylparaben, propylparaben, camphor, benzoin, chromium hydroxide green, menthol, ceresin and iron oxides.

16. The method of claim 11, wherein said corticosteroid and said moist, non-viscous, semi-solid gelatinous paste are admixed prior to application to the affected area.

17. The method of claim 11, wherein said affected area is covered with an occlusive for at least 72 hours, said occlusive being replaced at least daily.

18. The method of claim 11, wherein said composition is an admixture of a corticosteroid; a pharmaceutically accepted carrier; and a moist, non-viscous, semi-solid gelatinous paste.

19. The method of claim 18, wherein said past includes at least one ingredient selected from the group consisting of mineral oil, lanolin, beeswax, cetyl esters, zinc oxide, witch hazel, propylene glycol, cocoa butter, glycerin, sd alcohol 40, boric acid, petrolatum, methylparaben, propylparaben, camphor, benzoin, chromium hydroxide green, menthol, ceresin and iron oxides.

20. The method of claim 18, wherein said composition further includes a penetrating enhancer.
